(19) United States
(12) Patent Application Publication
(10) Pub. No.: US 2026/0232467 A1
(43) Pub. Date: Aug. 13, 2026

(54) CRIMPER APPARATUS AND METHODS OF USE

(71) Applicant: Edwards Lifesciences Corporation, Irvine, CA (US)

(72) Inventor: Michael C. Murad, Lake Mathews, CA (US)

(73) Assignee: Edwards Lifesciences Corporation, Irvine, CA (US)

(21) Appl. No.: 19/637,924

(22) Filed: Apr. 2, 2026

Related U.S. Application Data
(63) Continuation of application No. PCT/US2024/050909, filed on Oct. 11, 2024.
(60) Provisional application No. 63/590,071, filed on Oct. 13, 2023.

(51) Int. Cl. A61F 2/95; A61F 2/24

(52) U.S. Cl. CPC A61F 2/9524; A61F 2/2433

(57) ABSTRACT

Crimper apparatus for crimping a prosthetic for crimping a prosthetic heart valve from a radially expanded state to a radially compressed state and associated methods of use are disclosed. The crimper apparatus includes a variable diameter crimping aperture that can be transitioned from a larger diameter to a smaller diameter via movement of an actuator from a first position to a second position. The crimper apparatus includes an indicator mechanism for indicating that the actuator is in the second position. The indicator mechanism includes an electrical circuit that is closed when the actuator is in the second position by bringing a first electrical contact on a stop member into communication with a second electrical contact on the actuator, and thereby activates an indicator device.

### CROSS-REFERENCE TO RELATED APPLICATIONS

This application is a continuation of PCT Application No. PCT/US2024/050909, filed October 11, 2024, which claims the benefit of U.S. Provisional Patent Application No. 63/590,071, filed October 13, 2023, the entire contents of each of which are incorporated herein by reference.

### FIELD

The present disclosure relates to crimper apparatus for crimping prosthetic heart valves from a radially expanded state to a radially compressed state including, for example, crimper apparatus having a handle that is moveable between a first position to a second position and an indicator mechanism for indicating that the handle is in the second position.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Balloon-expandable prosthetic valves are typically crimped from an initial large diameter to a smaller diameter to put them into the crimped state for advancement to a treatment site in the body. Before crimping, a balloon expandable prosthetic valve is typically placed over an inflatable balloon on a catheter shaft. In cases where the prosthetic valve was manufactured in its fully crimped diameter, the prosthetic valve is expanded and then crimped on the balloon. Once delivered to the implantation site, the balloon can be inflated to expand the prosthetic valve to its fully functional size. Self-expanding prosthetic valves are typically also crimped to a smaller diameter and are then inserted into a sheath to retain the self-expandable valve in the crimped state. After placement in the body, the sheath is withdrawn, and the prosthetic valve expands inside the body.

Crimper apparatus for crimping a prosthetic valve can include a plurality of circumferentially arranged moveable segments that define a variable diameter crimping aperture. When a prosthetic valve is placed in the crimping aperture, the moveable segments are moved radially inwardly to decrease the diameter of the crimping aperture, thereby radially compressing the prosthetic valve from an initial larger diameter to a smaller diameter. Such crimper apparatus may include a manually rotatable handle for controlling transition of the crimping aperture between a first (larger) diameter and a second (smaller) diameter.

### SUMMARY

Described herein are exemplary prosthetic heart valves, crimper apparatus for radially compressing the prosthetic heart valves onto or for use with a delivery apparatus, and associated methods for crimping the prosthetic heart valves. The disclosed crimper apparatus and methods can, for example, provide feedback to a user to indicate that the prosthetic heart valve has been fully or sufficiently radially compressed. For example, the crimper apparatus can include an electrical circuit within or on a body of the apparatus, a first electrical contact in communication with the electrical circuit and disposed or positioned on a handle or actuator of the apparatus, a second electrical contact in communication with the electrical circuit and disposed or positioned on a stop member, and an indicator mechanism in communication with the electrical circuit. When the handle or actuator is moved from a first position to a second position to radial compress the prosthetic valve, the first electrical contact is brought into communication with the second electrical contact to close the circuit and activate the indicator mechanism to alert an operator or user that the prosthetic heart valve has been fully or sufficiently radially compressed. As such, the devices and methods disclosed herein can, among other things, overcome one or more of the deficiencies of typical crimper apparatus and methods of use thereof.

A prosthetic heart valve can comprise a frame and a valvular structure coupled to the frame. In addition to these components, a prosthetic heart valve can further comprise one or more of the components disclosed herein.

According to an aspect of the disclosure, a crimper apparatus can comprise a crimping aperture and an actuator that is moveable from a first position to a second position for transitioning the crimping aperture from a first diameter to a second smaller diameter.

In some examples, the crimper apparatus can include an indicator mechanism for indicating when the actuator is in the second position.

In some examples, the actuator is a handle.

In some examples, the actuator is a lever.

In some examples, the actuator is a knob.

In some examples, the actuator can include a first electrical contact.

In some examples, the crimper apparatus can include a stop member on a base or a frame of the crimper apparatus, and the stop member can include a second electrical contact.

In some examples, the stop member can be a projection extending from the base or the frame of the crimper apparatus.

In some examples, the actuator can include a first abutment surface for abutment to a tip or an end portion of the stop member, which forms a second abutment surface.

In some examples, the first abutment surface can be a receptacle configured to receive the second abutment surface.

In some examples, the first electrical contact is located on the first abutment surface and the second electrical contact is located on the second abutment surface.

In some examples, the stop member is detachable.

In some examples, a length or a height of a stop member can be selected to limit and/or enable closing of the crimping aperture to a specified diameter.

In some examples, the first and the second electrical contacts can be brought into communication to activate an indicator device.

In some examples, when the in first position, the actuator is spaced apart from the second electrical contact of the stop member and, in the second position, the first electrical contact of the actuator contacts the second electrical contact.

In some examples, the first and the second electrical contacts are in communication with an electrical circuit that extends through a body, a housing, and/or a frame of the crimper apparatus.

In some examples, the electrical circuit can include a circular lead portion that extends through or over a circular portion of a housing of the crimper apparatus.

In some examples, the electrical circuit is in communication with a power source.

In some examples, the power source can be an on-apparatus power source stored in a body, a frame, and/or a base of the crimper apparatus.

In some examples, the power source can be an off-apparatus power source.

In some examples, the indicator mechanism can include a switch for turning the indicator mechanism on and off.

In some examples, an indicator device in communication with the electrical circuit can be configured for haptic, visual, and/or auditory feedback.

In some examples, the indicator device includes one or more lights.

In some examples, the one or more lights can be positioned on a surface of the actuator.

In some examples, the crimper apparatus can include a housing and circumferentially arrayed nesting jaw members radially moveable within the housing, each of the jaw members having an inner end forming a crimping surface which combines with the crimping surfaces on others of the jaw members to form the crimping aperture.

In some examples, movement of the actuator from the first position to the second position produces radial movement of the jaw members to transition the crimping aperture from the first diameter to the second smaller diameter.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view illustrating one embodiment of a crimper apparatus.

FIG. 2 is a side elevation view of the crimper apparatus of FIG. 1, illustrating the crimping aperture when the jaw members of the crimper apparatus are in their open positions.

FIG. 3 is a side elevation view of the crimper apparatus of FIG. 1, illustrating the crimping aperture when the jaw members are in their closed positions.

FIG. 4 is an exploded, perspective view showing the components of the crimper apparatus of FIG. 1.

FIG. 5 is a side elevation view of the crimper apparatus of FIG. 1 with a side portion of the base removed illustrating the spiral tracks, channels, and camming members configured for moving the jaw members.

FIG. 6A shows an exemplary prosthetic heart valve in a radially expanded state.

FIG. 6B shows the valve of FIG. 6A in a radially compressed state.

FIG. 6C is a cross-sectional view of a distal end of an exemplary delivery device that includes a valve in a radially compressed state mounted over an inflatable balloon.

FIG. 7A is a perspective view of another exemplary crimper apparatus including an indicator mechanism, showing a handle of the crimper apparatus in a first position.

FIG. 7B is a perspective view of the crimper apparatus of FIG. 7A, showing the handle in a second position, which activates the indicator mechanism.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures.

As introduced above, crimper apparatus for crimping a prosthetic valve can include a plurality of circumferentially arranged moveable segments that define a variable diameter crimping aperture. When a prosthetic valve is placed in the crimping aperture, the moveable segments can be moved radially inwardly to decrease the diameter of the crimping aperture, thereby radially compressing the prosthetic valve from an initial larger diameter to a smaller diameter so that the prosthetic valve can be reduced to a diameter for transcatheter delivery within anatomy of a patient. Such crimper apparatus may include a manually operable handle or actuator for controlling transition of the crimping aperture between a first (larger) diameter and a second (smaller) diameter.

During use of a manually operated crimper apparatus, a user or operator must provide exert a force on the handle or actuator to sufficiently or fully radially compress the prosthetic valve. It may be difficult for an operator to determine or feel that the prosthetic valve has been sufficiently radially compressed. Thus, the crimping device is subject to operator error, which may, in some examples, result in the prosthetic valve remaining partially radially expanded at a diameter that hinders or prevents transcatheter delivery thereof. For example, when the prosthetic valve remains partially radially expanded, excessive force may be required for pushing the prosthetic valve through a delivery sheath or insertion through the delivery sheath may be limited or prevented.

Described herein are exemplary crimper apparatus and methods for radially compressing prosthetic heart valves that can, among other things, overcome one or more of the deficiencies of typical crimper apparatus and methods of use thereof. For example, the disclosed crimper apparatus and methods can, for example, provide feedback to a user to indicate that the prosthetic heart valve has been fully or sufficiently radially compressed. For example, the crimper apparatus can include an electrical circuit within or on a body of the apparatus, a first electrical contact in communication with the electrical circuit and disposed or positioned on a handle or actuator of the apparatus, a second electrical contact in communication with the electrical circuit and disposed or positioned on a stop member, and an indicator mechanism in communication with the electrical circuit. When the handle or actuator is moved from a first position to a second position to radial compress the prosthetic valve, the first electrical contact is brought into communication with the second electrical contact to close the circuit and activate the indicator mechanism to alert an operator or user that the prosthetic heart valve has been fully or sufficiently radially compressed.

### Examples of the Disclosed Technology

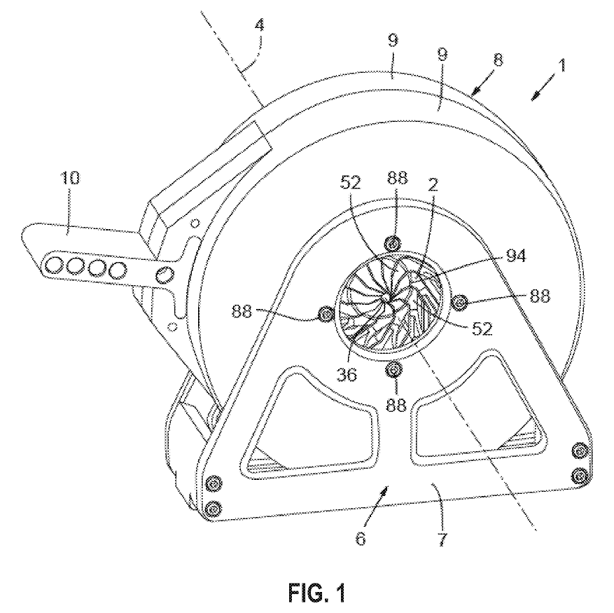

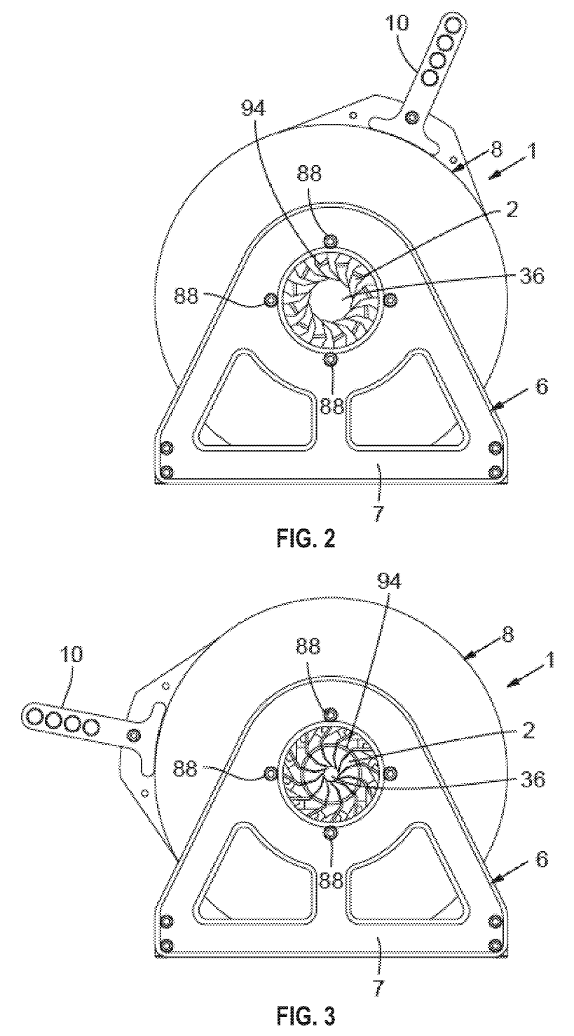

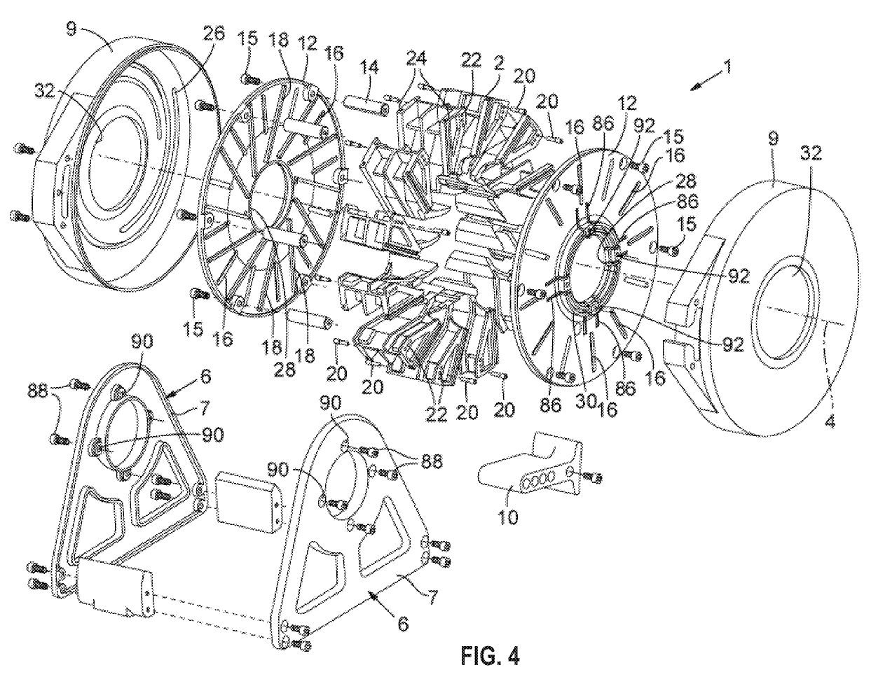

With reference to FIG. 1, an exemplary prosthetic valve or stent crimper apparatus 1 (also referred to herein as a “crimper apparatus”) is shown. The crimper apparatus 1 can include a plurality of nested jaw members 2 (for example, twelve nested jaw members) arranged around a central axis 4 of the jaw members. The jaw members 2 are shown in a “closed” position in FIG. 1 defining a variable-sized crimping aperture 36 (FIGS. 2 and 3) between their inner ends. In some examples, the number of jaw members can range between 6 and 12. In other examples, a greater or fewer number of jaw members can be used. In the illustrated example, each inner plate 12 has one channel 16 for each jaw member and each jaw member 2 has two guide slots 24. In examples including fewer jaw members, each jaw member can be larger to provide the necessary contributory crimping surface in the aperture. For example, each jaw member can have a greater surface area at an end region thereof forming a portion of the aperture.

The crimper apparatus 1 can further include a stationary frame 6 which acts as a stand or base. The jaw members 2 can be located within a rotating portion comprising a two-part outer housing 8 comprising end caps 9. An actuator in the form of a lever or handle 10 can be connected to and extend outwardly from the end caps 9. Each end cap 9 of the outer housing 8 can comprise a generally disk-shape with a radially-oriented circular wall and an annular outer rim extending toward the opposite housing part. In some examples, the outer rims of the end caps 9 can contact one another and surround the internal mechanism circumferentially. In other examples, the outer rims of the end caps 9 do not contact each other, for example, contacting one or more other outer housing components, for example, a shell.

FIGS. 2 and 3 show side views of the crimper apparatus 1. FIG. 2 shows the crimper apparatus 1 in an “open” position, with the jaw members 2 moved to their radially outward position such that the aperture 36 has a relatively large diameter for receiving a prosthetic valve in a fully radially expanded state. In other words, when in the “open” or radially expanded position, the jaw members can be moved radially outward from each other far enough to allow an annular body in an expanded state to be inserted into the crimping aperture. FIG. 3 shows the jaw members 2 in a “closed” or constricted position, such that the aperture 36 has a smaller diameter for crimping the prosthetic valve. In other words, when in the “closed” or radially contracted position, the jaw members can be moved radially inward toward each other to radially compress the annular body in the crimping aperture.

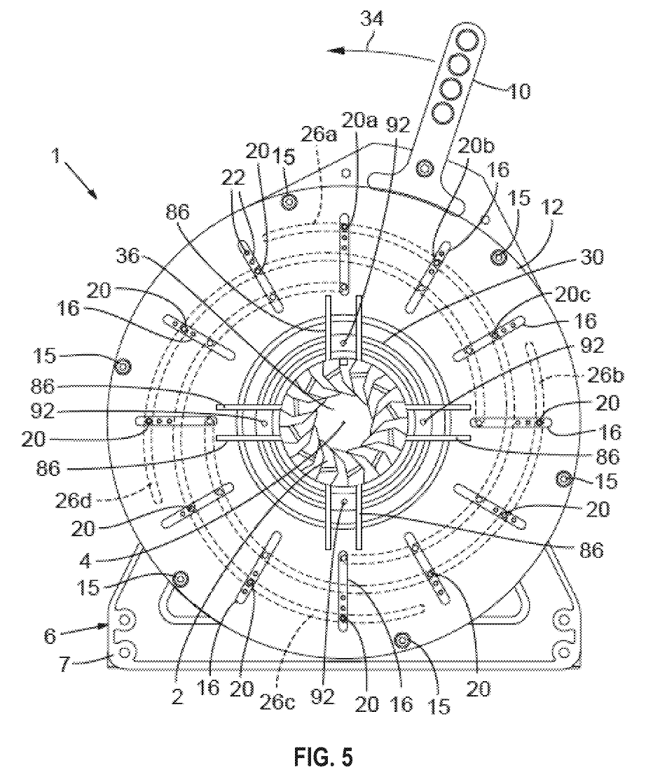

FIG. 4 shows an exploded view of the crimper apparatus 1. As can be seen in FIG. 4, the jaw members 2 can be arranged around the central axis 4 and can be flanked on both sides by two inner or end plates 12. The inner plates 12 can be disk-shaped and can be connected to each other by a plurality of spacers 14 (for example, six spacers) that are spaced around the circumferential edge of the inner plates 12 and surround the jaw members 2 circumferentially. The spacers 14 can be secured to the inner plates 12 by a plurality of screws 15, each of which extends through a corresponding opening in one of the plates 12 and is tightened into a corresponding threaded opening in one end of the corresponding spacer 14. The assembly of the two inner plates 12 and the spacers 14 can define a generally cylindrical cavity that contains the jaw members 2. The axial dimensions of the jaw members 2 and lengths of the spacers 14 can be such that the jaw members 2 are axially and rotatably constrained between the inner faces of the inner plates 12, but have sufficient clearance to enable sliding or radial movement of the jaw members 2 therein.

Each of the inner plates 12 can further comprise a plurality of spoke-like channels 16 (for example, twelve channels), one for each of the jaw members 2, which are evenly spaced and arranged on radial lines extending outward from the center of the inner plates 12. Each of the inner plates 12 can also include a plurality of guide tabs 18, each corresponding to and offset from a respective channel 16. Each of the jaw members 2 can be provided with or include a pair of camming members or pegs 20, where one camming member or peg projects outwardly from each side of a respective jaw member. In some examples, the camming members 20 can be located in one of three holes 22 on each side of the jaw member 2, the holes 22 being on a radial line from the center axis 4 and being located near the radially outermost edge of a respective member 2. Each jaw member 2 can further include a pair of radially extending guide slots 24 (one on each side of the jaw member) that are offset from the camming members 20. The guide slots 24 can engage with the guide tabs 18 on the inner plates 12, while the camming members 20 can pass through the channels 16 and interact with spiral channels 26 located in the end caps 9 of the outer housing 8.

Each inner plate 12 can further include a central hole or aperture 28 with an axially-oriented rim 30 extending away from the jaw members 2. The rim 30 can extend through a center hole or aperture 32 of an adjacent end cap 9, and can engage an adjacent side portion 7 of the stand 6, such that the outer housing 8 is between the side portions 7 of the stand 6 and the inner plates 12, and can rotate relative to the inner plates 12. Each of the inner plates 12 can be secured to a respective side portion 7 of the stand 6 with a plurality of screws 88 that extend through corresponding openings 90 in the side portion 7 and are tightened into corresponding threaded openings 92 in the inner plate 12. Accordingly, rotating the lever 10 can cause corresponding rotation of the outer housing 8 relative to the stand 6 and the inner plates 12.

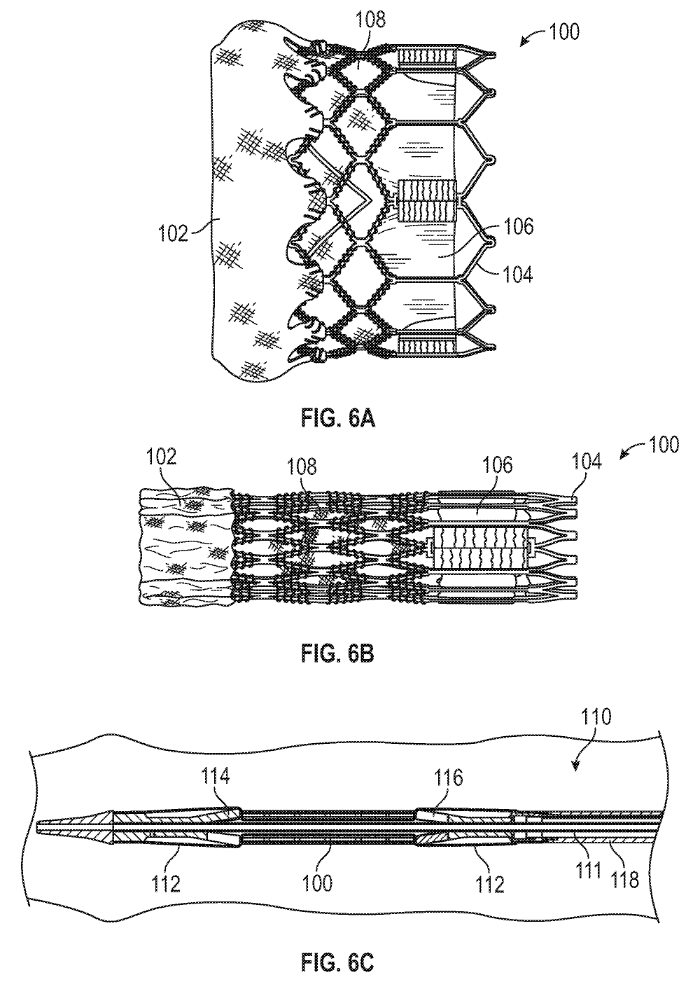

FIG. 5 shows a side view of the crimper apparatus 1 with one of the side portions 7 of the stationary frame 6 (that is, the closest side portion 7) and one of the end caps 9 of outer housing 8 (that is, the closest end cap 9) removed for illustrative purposes. FIG. 5 also indicates in phantom the position of the four independent spiral channels or tracks 26a, 26b, 26c, 26d located on an inner surface of the removed end cap 9, and indicates the positions of the channels 16 in the adjacent inner plate 12 relative to the spiral channels 26a, 26b, 26c, 26d. The holes 22 that are not occupied by a camming member 20 are also indicated in FIG. 5 for illustrative purposes.

As shown, camming members 20 on the jaw member 2 can pass through the channels 16 and into the spiral channels 26a, 26b, 26c, 26d. Each spiral channel 26a, 26b, 26c, 26d can interact with three consecutive or adjacent jaw members 2 via their respective camming members 20. For example, as shown in FIG. 5, progressing in a clockwise direction starting at a 12 o’clock position, the first of the three consecutive or adjacent jaw members 2 has a camming member 20a in the radially outermost hole 22 and extending into channel 26a, the second (middle) jaw member has a camming member 20b in the middle hole 22 and extending into channel 26a, and the third jaw member has a camming member 20c in the radially innermost hole 22 and extending into channel 26a. Each of the remaining channels 26b, 26c, and 26d similarly interact with a respective set of three consecutive or adjacent jaw members 2.

Upon rotation (for example, counter-clockwise rotation) of the outer housing 8, the spiral channels 26a, 26b, 26c, 26d can apply a generally radially inward camming force to the camming members 20 and the jaw members 2 attached thereto. In other words, the constrained movement of the camming members 20 along the spiral channels 26a, 26b, 26c, 26d can produce radial motion of the jaw members 2 toward the central axis 4. Further, the motion of the jaw members 2 can be constrained both by the interaction of the camming members 20 with the channels 16, as well as by the guide slots 24 sliding over the guide tabs 18 (FIG. 4). As can be seen in FIG. 5, when the handle 10 is rotated in the counterclockwise direction of the arrow 34, the rotation of the handle can cause the outer housing 8 rotate, which thereby can cause the spiral tracks 26 to rotate. The rotational movement of the spiral tracks 26 can result in the camming members 20 driving movement of the jaw members 2 radially inward to reduce the diameter of the crimping aperture 36 (FIG. 5).

It will be appreciated that the foregoing crimper apparatus is exemplary, and a crimper apparatus can include additional, alternate, or fewer features. For example, a crimper apparatus can include only one of the end plates 12 having the channels 16 and/or the guide tabs 18, and the jaw members 2 can include only one corresponding camming member 20 and/or guide slot 24. Additional and alternate features of exemplary crimper apparatus are disclosed in U.S. Patent No. 7,530,253 and U.S. Patent Application Publication No. 2016/0199184, which are each incorporated herein by reference.

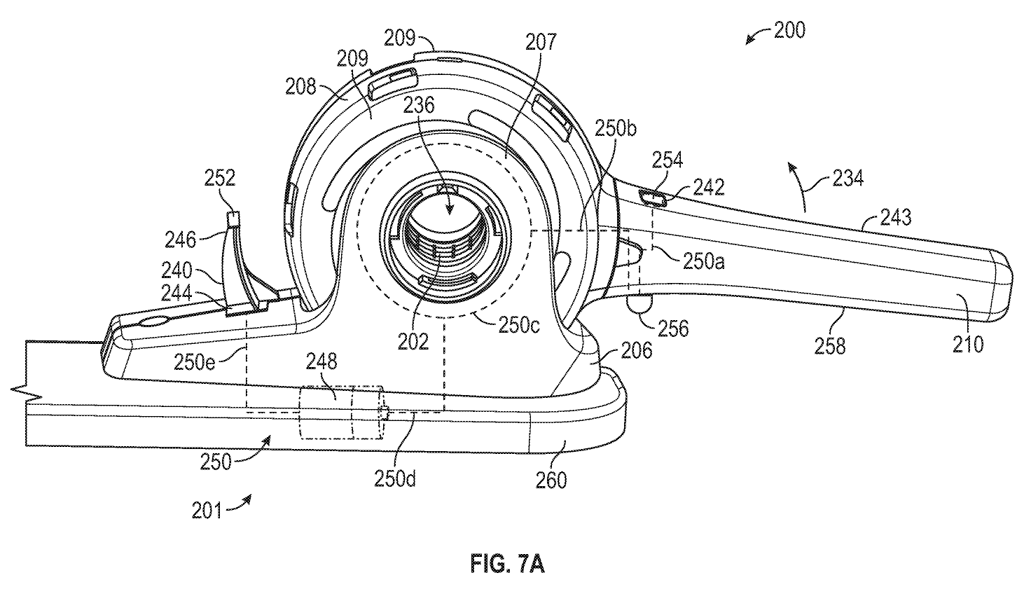

FIG. 6A-6C show an exemplary prosthetic heart valve 100 that can be used with the herein disclosed crimping devices and crimping methods. The valve 100 is just one example, and many other implantable device can be used as well, including stents, valves, frames, and any other radially compressible device. Such device can include annular frames that are radially compressible to be mounted on a delivery device and later radially expandable within a patient’s body. Such frames can include self-expandable frames (for example, made of superelastic materials) and balloon-expandable frames (for example, made of cobalt chromium or stainless steel). The valve 100 can be configured for placement at a native aortic valve region of heart, while other subject devices can be configured for placement at other heart locations or other parts of the body. In the example of FIG. 6A, an outer skirt 102 is positioned at a blood inflow end of the valve 100, with the opposite end being a blood outflow end of the valve.

As can be seen in FIGS. 6A-6B, the prosthetic heart valve 100 can include a frame 104, the outer skirt 102 disposed on an outer surface of the frame 104, a valve structure 106 supported within the frame 104, and an inner skirt 108 disposed on an interior surface of frame 104. In some examples, one or more of the foregoing components can be excluded. For example, a prosthetic valve can exclude an inner skirt and/or an outer skirt. In some examples, a prosthetic valve can include additional components. For example, a prosthetic valve can additionally include a secondary frame exterior of the frame supporting the valve structure.

The frame 104 can be made of any of various suitable plastically-expandable materials (for example, stainless steel, etc.) or self-expanding materials (for example, Nitinol) as known in the art. When constructed of a plastically-expandable material, the frame 104 (and thus the valve 100) can be crimped to a radially compressed state on a delivery catheter and then expanded inside a patient by an inflatable balloon or equivalent expansion mechanism. When constructed of a self-expandable material, the frame 104 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 104) include, metal alloys, polymers, or combinations thereof. Example metal alloys can comprise one or more of the following: nickel, cobalt, chromium, molybdenum, titanium, or other biocompatible metal. In some examples, the frame 104 can comprise stainless steel. In some examples, the frame 104 can comprise cobalt-chromium. In some examples, the frame 104 can comprise nickel-cobalt-chromium. In some examples, the frame 104 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

FIG. 6A shows the prosthetic valve 100 (comprising an annular body) in a radially expanded state, while FIG. 6B shows the prosthetic valve 100 in a radially compressed or crimped state. In the crimped state, the prosthetic valve 100 can have a smaller radial dimension (that is, a smaller outer diameter) and a longer axial dimension (that is, a longer axial length) relative to the radially expanded state. In some examples, portions of the prosthetic valve 100 can have slightly different outer diameters when in the crimped state. For example, in some circumstances the axial zone including the outer skirt 102 can be slightly bulkier and have a larger outer diameter compared to other portions of the valve 100.

FIG. 6C shows a distal end portion of an exemplary delivery device 110 with the prosthetic valve 100 crimped thereon. Specifically, in the example of FIG. 6C, the prosthetic valve 100 can be crimped onto an inner shaft 111 over an inflatable balloon 112 between a distal shoulder 114 and a proximal shoulder 116. The shoulders 114 and 116 can be positioned around the shaft 111 within distal and proximal portions of the balloon 112. In some examples, when the prosthetic valve 100 is crimped onto the delivery device 110, the shoulders 114, 116 can protect or shield the distal and proximal ends of the prosthetic valve 100 to help prevent the ends of the valve from snagging on a catheter wall, a vessel wall, or other structure during transcatheter delivery of the prosthetic valve 100. In some examples, the shoulders 114, 116 can also limit or prevent axial migration of the prosthetic valve 100 relative to the inner shaft 11. In some examples, crimping the distal end and/or the proximal end of the prosthetic valve 100 to a small outer diameter than the rest of the valve can also limit or prevent snagging or axial migration of the prosthetic valve 100 during transcatheter delivery thereof. When the prosthetic valve 100 is delivered to an implantation location (such as, for example, a native aortic valve), the balloon 112 can be inflated to radially expand the prosthetic valve 100 and implant it in the native aortic valve (or other target location).

It will be appreciated that the foregoing prosthetic valve and delivery device are exemplary, and other prosthetic valves and/or delivery devices can be utilized with the crimper apparatus disclosed herein.

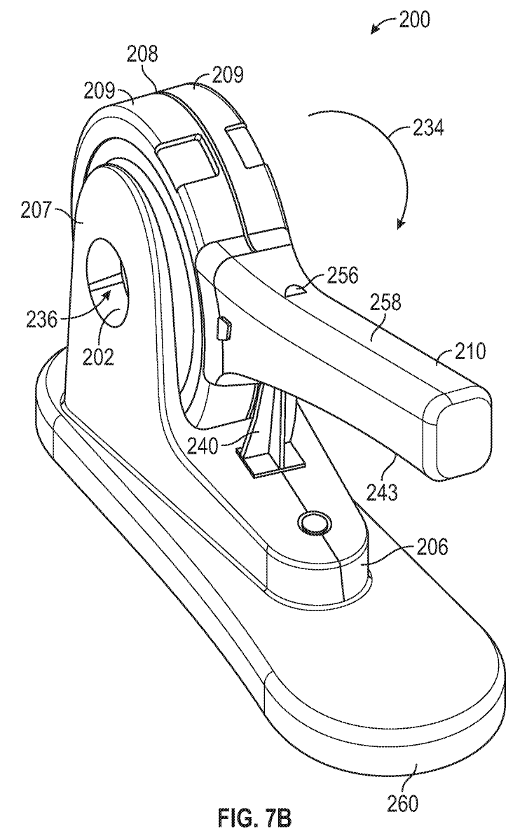

FIGS. 7A and 7B illustrate another exemplary crimper apparatus 200 including an indicator mechanism 201 for providing feedback to a user to indicate a prosthetic valve is sufficiently or fully radially compressed, in accordance with the present disclosure. The crimper apparatus can, in some examples, have a configuration similar to that of the crimper apparatus 1, and can include one or more of the features discussed above with reference to FIGS. 1-5. In other examples, the crimper apparatus 200 can include fewer, additional, or alternate features relative to the crimper apparatus 1.

As can be seen in FIG. 7A, the crimper apparatus 200 can include a plurality of nested jaw members 202 (for example, twelve nested jaw members) arranged around a central axis of the jaw members. The jaw members 202 are shown in an “open” position in FIG. 7A and can define a variable-sized crimping aperture 236 between their inner ends. The crimper apparatus 200 can further include a stationary frame 206 (including opposing side portions or members 207) which acts as a stand or base and a two-part outer housing 208 comprising end caps 209. An actuator 210 in the form of a lever or handle can be connected to and extend outwardly from the end caps 209.

The crimper apparatus 200 can be operated similarly to the crimper apparatus 1 for crimping a prosthetic valve (for example, the prosthetic valve 100) from an initial larger diameter to a smaller diameter to transition the valve from a radially expanded state to a radially compressed state. For example, the actuator 210 can be rotated in a direction 234 (for example, counterclockwise direction as shown in FIG. 7A) from a first position where the jaw members 202 are open (FIG. 7A) to a second position where the jaw members 202 are “closed” (FIG. 7B) to reduce a diameter of the crimping aperture 236 and radially compress a prosthetic valve disposed therein.

In addition to the features discussed above with respect to the crimper apparatus 1, the crimper apparatus 200 can further include a stop member 240 extending upwardly from a portion of the frame 206. Specifically, the stop member 240 can extend from a portion of the frame 206 that is on an opposing side of the base relative to actuator 210 when it is in the first position, and is on a same side of the base as the actuator 210 when it is in the second position. In use, the stop member 240 can be configured to prevent further rotation of the level 210 in the counterclockwise direction by abutting a close stop surface 242 formed in or on a first surface 243 of the actuator 210. In some examples, the first surface 243 is an upper surface of the actuator 210 when the actuator is in the first position, and is a lower surface of the actuator 210 when the actuator is in the second position. In some examples, when the close stop surface 242 contacts or is abutted to the stop member 240, the actuator 210 can be in the second position.

In the example of FIGS. 7A and 7B, the stop member 240 member is formed by two intersecting walls (for example, two intersecting walls that are perpendicular relative to each other) and has an overall tapered shape including a wider base portion 244 and a distal end or tip portion 246. In some examples, the close stop surface 242 can be sized and shaped to receive and/or abut the distal tip portion 246 of the stop member 240. For example, the close stop surface 242 can be a receptacle or indentation sized and shaped to receive and abut the distal tip portion 246.

It will be appreciated that the stop member can have a different configuration than the illustrated example. In some examples, the stop member can be a cylindrical member. In some examples, the stop member can be a cuboid member. In some examples, the stop member can be a plate or shelf or a flat surface. In some examples, the stop member can be a round or partial sphere member. In some examples, the stop member can include a channel and/or a receptacle or indentation configured to receive a portion of the lever or handle or a projection on a surface of the lever or handle.

In some examples, the stop member can be removeable and/or replaceable. In some examples, stop members having varying configurations, for example, stop members of various heights, can be selectively removeable and attachable to the frame. In such examples, a stop member can be selected for use with the crimper apparatus to control a degree of closing of the crimping aperture (for example, to enable closing of the crimping aperture to a specified diameter). For example, a first stop member with a greater height can limit closing of the aperture to a larger diameter, while a second stop member with a shorter height can allow closing of the aperture to a smaller diameter. In some examples, the removable stop members can be selected for use with different types and/or sizes of prosthetic valves. In some examples, the removable stop members can be selected for under-compressing or over-compressing a prosthetic valve.

It will be appreciated that the close stop surface can have a complementary configuration and/or shape for interaction with and/or abutment to a distal portion or other upward facing portion of the stop member. In other words, the stop member comprises a first abutment surface having the first electrical contact disposed thereon, and the handle comprises a second abutment surface having the second electrical contact disposed thereon, the second abutment surface having a complementary shape to the first abutment surface.

As shown in FIG. 7A, the indicator mechanism 201 can include an electrical circuit 250 (for example, an electrically conductive pathway) in communication with the power source 248 (for example, an on-board battery or an external power source). The electrical circuit 250 can be in further communication with a first electrical contact 252 disposed on the distal end portion 246 of the stop member 240, a second electrical contact 254 disposed on or within the close stop surface 242, and an indicator 256 (for example, an indicator device or an electrically actuatable indicator such as, for example, a visual indicator, an auditory or aural indicator, and/or a haptic feedback indicator). The electrical circuit can be formed by one or more conductive materials comprising, for example, elongated aggregates of particles or fibers in electrical contact with each other. The electrical circuit materials can be preformed (for example, wires or metallic strips) and attached to the crimper apparatus and/or can be a conductive material flowed or deposited onto a surface along the pathway.

The electrical circuit 250 can be in an open state (preventing electrical current therethrough) when the actuator 210 is in the first position (FIG. 7A), as well as intermediate positions of the actuator 210 from the first position and prior to reaching the second position. When the actuator 210 is in the second position (FIG. 7B), the first electrical contact 252 and the second electrical contact 254 can be brought into communication to transition the electrical circuit 250 to a closed state (enabling electrical current therethrough). Closing of the electrical circuit can activate the indicator device 256, thereby a signal or a cue to a user that the actuator 210 is in the second position and a prosthetic valve disposed within the aperture 236 is in the sufficiently or fully radially compressed state.

In some examples, the electrical circuit can further include a switch (not shown) for turning the indicator mechanism on and off by, for example, enabling and disabling closing of the electrical circuit or by enabling and disabling communication between the power source and the electrical circuit. In such examples, when the switch is in a closed (“on”) position, the first and second electrical contacts can be brought into communication to close electrical circuit and provide a current therethrough for activation of the indicator device. However, when the switch is in an open (“off”) position, the electrical circuit can remain open or have no electrical current even when the first and second electrical contacts are brought into communication, and activation of the indicator device can be prevented. Incorporating an on-off switch is advantageous in that the crimper apparatus 200 can be stored (prior to use) with the actuator 210 in the second position contacting the stop member 240 (FIG. 7B) without draining an onboard battery when the switch is in the off position.

In the present example, the indicator device 256 comprises an LED light disposed on an opposing second surface 258 of the actuator 210 relative to the first surface 243. In some examples, the second surface 258 is a lower surface of the actuator 210 when the actuator is in the first position, and is an upper surface of the actuator 210 when the actuator is in the second position. Thus, in the present example, the indicator device 256 is positioned to be observed or visually detected by a user or operator of the device when the actuator 210 is in the second position. In some examples, the indicator device 256 can be a light in a different position, such as an indicator light positioned on the frame 206 or an external, off-crimper apparatus indicator light. In some examples, the indicator device 256 can be a different types of visual indicator such as, a screen or display device. As discussed above, in some examples, the indicator device 256 can be or can additionally include an auditory or aural indicator (for example, an alarm or buzzer) and/or a haptic feedback indicator (for example, a device or mechanism configured to apply a force or vibration to the actuator 210 when it is in the second position).

As shown in FIG. 7A, in some examples, the electrical circuit 250 can include a first lead portion 250a, a second lead portion 250b, a third lead portion 250c, a fourth lead portion 250d, and a fifth lead portion 250e. The first lead 250a can be in communication with the second electrical contact 254 and the indicator device 256, and can extend through or over or be deposited on a surface (for example, an interior surface) the actuator 210. The second lead 250b can be in communication with the indicator device 256 and the third lead portion 250c, and can extend through or over or be deposited on a surface (for example, an interior surface) the actuator 210.

The third lead portion 250c can be a disc contact or a circular lead configured for communication with the second lead 250b (disposed in or on the actuator 210) and the fourth lead 250d (disposed in or on the frame 206). In some examples, the circular lead 250c can be mounted to and/or deposited on a portion of the housing 208 (such as, for example, on an interior surface of one of the end caps 209), and can be stationary relative to the second lead 250b and rotatable relative to the fourth lead 250d. In other examples, the disc contact or circular lead 250c can be mounted to and/or deposited on a portion of the frame 206 (such as, for example, on an interior surface of one of the side portions 207), and can be stationary relative to the fourth lead 250d, while the second lead 250b is rotatable relative to the disc contact or circular lead 250c. In the foregoing examples, the disc contact or circular lead 250c can remain in communication with each of the second lead portion 250b and the fourth lead portion 250d during rotation of the actuator 210 between the first and second positions. Further, the second electrical contact 254 maintains communication with the electrical circuit 250 during rotation or the actuator 210 via the circular lead portion 250c.

As noted above, the fourth lead portion 250d is disposed in or on the frame 206, and can extend through or over or be deposited on a surface (for example, an interior surface) of a lower region one of the side portions 207 to the power source 248 for communication therewith. For example, the fourth lead portion 250d can be in communication with a first electrode of the power source 248. The fifth lead portion 250e can be in communication with the power source 248 (for example, a second electrode of the power source 248) and the first electrical contact 252, and can extend through or over or be deposited on a surface (for example, an interior surface) the frame 206 and can extend through or over or be deposited on a surface the stop member 240. For example, the fifth lead portion 250e can extend over a surface of the one of the walls of the stop member to the distal end portion 246 including the first electrical contact 252.

It will be appreciated that the configuration of the electrical circuit 250 is merely exemplary. In other examples, the electrical circuit can have a different pathway through the body of the crimper apparatus that provides a communication between the first electrical contact 252, the second electrical contact 254, the power source 248, and the indicator device 256, and forms an open circuit in the first position of the actuator 210 and a closed circuit in the second position of the actuator 210. For example, the circular lead can be excluded, and the second lead portion 250b can include an electrical contact that can be brought into communication with an electrical contact on the lead portion 250d when the handle is in the closed position. In other words, the electrical circuit can be closed by two pairs of electrical contacts being brought into communication, a first pair disposed on the handle and the stop member and a second pair disposed on the housing and the base.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations and examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve crimper apparatus for radially compressing an expandable prosthetic valve, the crimper apparatus comprising: a housing; a stop member having a first electrical contact disposed thereon, the first electrical contact coupled to an electrical circuit; a plurality of circumferentially arrayed nesting jaw members radially moveable within the housing, each of the jaw members having an inner end forming a crimping surface which combines with the crimping surfaces on others of the jaw members to form a crimping aperture that can transition from a first diameter to a second smaller diameter for crimping the expandable prosthetic valve; and a handle coupled to the housing, wherein the handle is moveable between a first position and a second position, wherein movement of the handle from the first position to the second position produces radial movement of the jaw members to transition the crimping aperture from the first diameter to the second smaller diameter, and wherein the handle has a second electrical contact disposed thereon and coupled to the electrical circuit; wherein the second electrical contact is positioned to, when the handle is in the second position, contact the first electrical contact of the stop member and activate an indicator device.

Example 2. The crimper apparatus of any example disclosed herein, particularly example 1, wherein the second electrical contact is positioned at a first surface of the handle, wherein the first surface is an upper surface of the handle when the handle is in the first position and is a lower surface of the handle when the handle is in the second position.

Example 3. The crimper apparatus of any example disclosed herein, particularly example 2, wherein the indicator device comprises a visual indicator device positioned at a second surface of the handle, wherein the second surface opposes the first surface, and wherein the second surface is a lower surface of the handle when the handle is in the first position and is an upper surface of the handle when the handle is in the second position.

Example 4. The crimper apparatus of any example disclosed herein, particularly examples 1-3, further comprising a base, wherein the housing is rotatably mounted to the base and a portion of the electrical circuit extends through or over the base.

Example 5. The crimper apparatus of any example disclosed herein, particularly example 4, wherein the stop member projects upwardly from the base.

Example 6. The crimper apparatus of any example disclosed herein, particularly example 4 or example 5, wherein the electrical circuit comprises a first circular lead portion.

Example 7. The crimper apparatus of any example disclosed herein, particularly example 6, wherein the first circular lead portion is disposed on a side member of the base, wherein the housing is rotatable relative to the first circular lead portion, and wherein the electrical circuit further comprises a second lead portion in communication with the second electrical contact and the first circular lead portion, the second lead portion configured to maintain contact with the first circular lead portion during rotation of the housing.

Example 8. The crimper apparatus of any example disclosed herein, particularly example 7, wherein the electrical circuit further comprises a third lead portion in communication with the first circular lead portion and the first electrical contact.

Example 9. The crimper apparatus of any example disclosed herein, particularly example 6, wherein the first circular lead portion is disposed on the housing, wherein first circular lead portion is rotatable relative to the base, and wherein the electrical circuit further comprises a second lead portion in communication with the first electrical contact and the first circular lead portion, the second lead portion configured to maintain contact with the first circular lead portion during rotation of the housing.

Example 10. The crimper apparatus of any example disclosed herein, particularly examples 1-9, wherein the stop member is a tapered member comprising a wider base portion and a narrower distal end portion, where the first electrical contact is disposed on the distal end portion.

Example 11. The crimper apparatus of any example disclosed herein, particularly example 10, wherein the handle comprises a close stop surface sized and shaped to receive the distal end portion and the first electrical contact, and wherein the second electrical contact is disposed on the close stop surface.

Example 12. A prosthetic valve crimper apparatus comprising: a plurality of crimping elements defining a crimping aperture that can transition from a first diameter to a second smaller diameter for crimping an expandable prosthetic valve; an electrical circuit; an indicator coupled to the electrical circuit; a stop member having a first electrical contact coupled to the electrical circuit; and a handle comprising a second electrical contact coupled to the electrical circuit, the handle operatively coupled to the crimping elements, wherein the handle is rotatable between a first position spaced from the stop member and a second position in which the second electrical contact of the handle contacts the first electrical contact of the stop member, wherein rotation of the handle from the first position to the second position moves the crimping elements and transitions the crimping aperture from the first diameter to the second smaller diameter; wherein, when the handle is in the second position, the first electrical contact and the second electrical contact are brought into communication to complete the electrical circuit and activate the indicator.

Example 13. The crimper apparatus of any example disclosed herein, particularly example 12, wherein the stop member comprises a first abutment surface having the first electrical contact disposed thereon, wherein the handle comprises a second abutment surface having the second electrical contact disposed thereon, and wherein the second abutment surface has a complementary shape to the first abutment surface.

Example 14. The crimper apparatus of any example disclosed herein, particularly example 13, wherein the second abutment surface comprises a receptacle for receipt of the first abutment surface.

Example 15. The crimper apparatus of any example disclosed herein, particularly example 13, wherein the first abutment surface comprises a receptacle for receipt of the second abutment surface.

Example 16. A prosthetic valve crimper apparatus comprising: a main body including a crimping aperture transitionable from a first diameter to a second smaller diameter for crimping an expandable prosthetic valve; a first electrical contact coupled to an electrical circuit; an actuator comprising a second electrical contact and operatively coupled to the main body, wherein movement of the actuator from a first position to a second position causes the crimping aperture to transition from the first diameter to the second smaller diameter, wherein, the in first position, the actuator is spaced from the first electrical contact and, in the second position, the second electrical contact of the actuator contacts the first electrical contact; and an electrically actuatable indicator in coupled to the electrical circuit; wherein, when the actuator is in the second position, the contact between the first electrical contact and the second electrical completes the electrical circuit and activates the electrically actuatable indicator.

Example 17. The prosthetic valve crimper apparatus of any example disclosed herein, particularly example 16, wherein the electrically actuatable indicator comprises a visual feedback indicator.

Example 18. The prosthetic valve crimper apparatus of any example disclosed herein, particularly example claim 16 or example 17, wherein the electrically actuatable indicator comprises a haptic feedback indicator.

Example 19. The prosthetic valve crimper apparatus of any example disclosed herein, particularly examples 16-18, wherein the electrically actuatable indicator comprises an auditory feedback indicator.

Example 20. A prosthetic valve crimper apparatus comprising: crimping means for radial compression of an expandable prosthetic valve from a larger diameter to a smaller diameter; actuator means for operating the crimping means, the actuator means moveable from a first position to a second position; and indicator means in communication with an electrical circuit that is closed when the actuator means is in the second position, the indicator means configured to generate a signal when the actuator means is in the second position.

The features described herein with regard to any example can be combined with other features described in any one or more of the other examples, unless otherwise stated. For example, any one or more of the features of one crimper apparatus can be combined with any one or more features of another crimper apparatus. As another example, any one or more features of one indicator mechanism can be combined with any one or more features of another indicator mechanism.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic valve crimper apparatus for radially compressing an expandable prosthetic valve, the crimper apparatus comprising:
a housing;
a stop member having a first electrical contact disposed thereon, wherein the first electrical contact is coupled to an electrical circuit;
a plurality of circumferentially arrayed nesting jaw members radially moveable within the housing, each of the jaw members having an inner end forming a crimping surface which combines with the crimping surfaces on others of the jaw members to form a crimping aperture that can transition from a first diameter to a second smaller diameter for crimping the expandable prosthetic valve; and
a handle coupled to the housing, wherein the handle is moveable between a first position and a second position, wherein the crimper apparatus is configured such that movement of the handle from the first position to the second position produces radial movement of the jaw members to transition the crimping aperture from the first diameter to the second smaller diameter, and wherein the handle has a second electrical contact disposed thereon and coupled to the electrical circuit;
wherein the second electrical contact is positioned to, when the handle is in the second position, contact the first electrical contact of the stop member and activate an indicator device.

2. The crimper apparatus of claim 1, wherein the second electrical contact is positioned at a first surface of the handle, wherein the first surface is an upper surface of the handle when the handle is in the first position and is a lower surface of the handle when the handle is in the second position.

3. The crimper apparatus of claim 2, wherein the indicator device comprises a visual indicator device positioned at a second surface of the handle, wherein the second surface opposes the first surface, and wherein the second surface is a lower surface of the handle when the handle is in the first position and is an upper surface of the handle when the handle is in the second position.

4. The crimper apparatus of claim 1, further comprising a base, wherein the housing is rotatably mounted to the base and a portion of the electrical circuit extends through or over the base.

5. The crimper apparatus of claim 4, wherein the stop member projects upwardly from the base.

6. The crimper apparatus of claim 4, wherein the electrical circuit comprises a first circular lead portion.

7. The crimper apparatus of claim 6, wherein the first circular lead portion is disposed on a side member of the base, wherein the housing is rotatable relative to the first circular lead portion, and wherein the electrical circuit further comprises a second lead portion in communication with the second electrical contact and the first circular lead portion, the second lead portion configured to maintain contact with the first circular lead portion during rotation of the housing.

8. The crimper apparatus of claim 7, wherein the electrical circuit further comprises a third lead portion in communication with the first circular lead portion and the first electrical contact.

9. The crimper apparatus of claim 6, wherein the first circular lead portion is disposed on the housing, wherein first circular lead portion is rotatable relative to the base, and wherein the electrical circuit further comprises a second lead portion in communication with the first electrical contact and the first circular lead portion, the second lead portion configured to maintain contact with the first circular lead portion during rotation of the housing.

10. The crimper apparatus of claim 1, wherein the stop member is a tapered member comprising a wider base portion and a narrower distal end portion, wherein the first electrical contact is disposed on the distal end portion.

11. The crimper apparatus of claim 10, wherein the handle comprises a stop surface sized and shaped to receive the distal end portion and the first electrical contact, and wherein the second electrical contact is disposed on the stop surface.

12. A prosthetic valve crimper apparatus comprising:
a plurality of crimping elements defining a crimping aperture that can transition from a first diameter to a second smaller diameter for crimping an expandable prosthetic valve;
an electrical circuit;
an indicator coupled to the electrical circuit;
a stop member having a first electrical contact coupled to the electrical circuit; and
a handle comprising a second electrical contact coupled to the electrical circuit, the handle operatively coupled to the crimping elements, wherein the handle is rotatable between a first position spaced from the stop member and a second position in which the second electrical contact of the handle contacts the first electrical contact of the stop member, wherein rotation of the handle from the first position to the second position moves the crimping elements and transitions the crimping aperture from the first diameter to the second smaller diameter;
wherein, when the handle is in the second position, the first electrical contact and the second electrical contact are brought into communication to complete the electrical circuit and activate the indicator.

13. The crimper apparatus of claim 12, wherein the stop member comprises a first abutment surface having the first electrical contact disposed thereon, wherein the handle comprises a second abutment surface having the second electrical contact disposed thereon, and wherein the second abutment surface has a complementary shape to the first abutment surface.

14. The crimper apparatus of claim 13, wherein the second abutment surface comprises a receptacle for receipt of the first abutment surface.

15. The crimper apparatus of claim 13, wherein the first abutment surface comprises a receptacle for receipt of the second abutment surface.

16. A prosthetic valve crimper apparatus comprising:
a main body including a crimping aperture transitionable from a first diameter to a second smaller diameter for crimping an expandable prosthetic valve;
a first electrical contact coupled to an electrical circuit;
an actuator comprising a second electrical contact and operatively coupled to the main body, wherein movement of the actuator from a first position to a second position causes the crimping aperture to transition from the first diameter to the second smaller diameter, wherein, in the first position, the actuator is spaced from the first electrical contact and, in the second position, the second electrical contact of the actuator contacts the first electrical contact; and
an electrically actuatable indicator in coupled to the electrical circuit;
wherein, when the actuator is in the second position, the contact between the first electrical contact and the second electrical contact completes the electrical circuit and activates the electrically actuatable indicator.

17. The prosthetic valve crimper apparatus of claim 16, wherein the electrically actuatable indicator comprises a visual feedback indicator.

18. The prosthetic valve crimper apparatus of claim 16, wherein the electrically actuatable indicator comprises a haptic feedback indicator.

19. The prosthetic valve crimper apparatus of claim 16, wherein the electrically actuatable indicator comprises an auditory feedback indicator.

20. The prosthetic valve crimper apparatus of claim 16, further comprising a base and a stop member that extends from the base, wherein the first electrical contact is disposed on a distal end portion of the stop member, wherein the actuator comprises a stop surface and the second electrical contact is disposed on the stop surface.
